(19) United States
(12) Patent Application Publication
(10) Pub. No.: US 2021/0196370 A1
(43) Pub. Date: Jul. 1, 2021

(54) NEUROSURGERY GUIDEWIRE WITH INTEGRAL CONNECTOR FOR SENSING AND APPLYING THERAPEUTIC ELECTRICAL ENERGY

(71) Applicant: Biosense Webster (Israel) Ltd., Yokneam (IL)

(72) Inventor: Assaf Govari, Haifa (IL)

(21) Appl. No.: 16/729,565

(22) Filed: Dec. 30, 2019

(51) Int. Cl. A61B 18/14; A61B 5/06; A61B 18/12

(52) U.S. Cl. CPC A61B 18/1485; A61B 2018/00077; A61B 18/1206; A61B 5/062

(57) ABSTRACT

A medical probe includes a guidewire, and a connector at a proximal end of the probe. The guidewire is configured to be inserted into an organ of a patient and includes one or more electrical devices fitted at a distal end of the guidewire, and two or more conductors, which connect the electrical devices to a proximal end of the guidewire. The conductors have proximal ends that are exposed at different positions along the proximal end of the guidewire. The connector is fitted with multiple conductive rings that are positioned to mate with the respective exposed proximal ends of the conductors, and to maintain electrical contact with the conductors while the guidewire is rotated.

### FIELD OF THE INVENTION

The present invention relates generally to invasive medical probes, and particularly to techniques to electromechanically connect to a distal end of a medical probe for insertion into a patient body.

### BACKGROUND OF THE INVENTION

Techniques to electromechanically connect a distal end of an invasive probe to an external module were previously proposed in the patent literature. For example, U.S. Pat. No. 6,355,005 describes an articulated guidewire for insertion into a blood vessel. The articulated guidewire includes a rotatable sensor cable, a sensor, a connector, and a satellite wire. The sensor cable has a proximal end and a distal end. A sensor connects to the sensor cable near the distal end and rotates with the sensor cable. The satellite wire attaches to the distal end of the sensor cable and holds the sensor cable in the blood vessel. The connector includes a ball and socket joint which aligns the satellite wire and the sensor cable at a variable angle. The sensor receives and converts into a corresponding electrical signal which is communicated through the slip rings and transmit/receive switch to a receiver.

As another example, European Patent 2,095,840 describes a female coupler for a guidewire that includes a disposable part and a non-disposable part. The disposable part includes a disposable tubular body having an open end and a closed end. At least one conducting spring is coupled with the disposable tubular body such that each of said at least one conducting spring has a portion thereof in contact with the inner wall of the disposable tubular body and a portion in contact with the outer wall of the disposable tubular body. A sheath is coupled with the disposable tubular body at the open end of the disposable tubular body, extending towards the closed end of the disposable tubular body, and a collet is coupled with the open end of the disposable tubular body for securing a male coupler within the disposable tubular body. The non-disposable part includes a non-disposable tubular body and at least one contact coupled with the non-disposable tubular body such that the circumference of the at least one contact encircles a portion of the circumference of the inner wall of the non-disposable tubular body. The disposable part is insertable into the non-disposable part, and at least one conducting spring is in electrical contact with the at least one contact when the disposable part is fully inserted into the non-disposable part.

U.S. Pat. No. 5,178,159 describes a guidewire assembly comprising a guidewire with first and second conductors which extend along the length thereof. The guide wire also comprises a flexible cable having first and second conductors which extend along the length thereof. A connector assembly is provided for interconnecting the flexible cable to said guide wire and interconnecting the conductors carried thereby. The connector assembly includes a male connector with a sleeve and a conductive core which is mounted in the sleeve. An insulator is mounted in the sleeve and insulates the conductive core from the sleeve. A conductive band is carried by the insulator and is spaced from the sleeve. The first and second conductors are disposed within the sleeve. The first connector is connected to the conductive core and the second conductor is connected to the conductive band. The connector assembly includes a female connector that has an inner conductive grip which has a cylindrical recess for receiving the conductive core and an outer conductor grip that has a cylindrical band which engages the portion extending forwardly of the inner conductive grip. An insulator is disposed between the inner and outer conductive grips. An insulating case is mounted on the outer conductive grip. First and second conductors are disposed within the case. The first conductor is connected to the inner conductive grip. The second conductor is connected to the outer conductive grip. The female connector receives the male connector and the first conductive grip receives the conductive core in the cylindrical recess of the first conductive grip. The second conductive grip receives the conductive band of the male connector by the cylindrical band receiving portion of the outer conductive grip engaging the band.

### SUMMARY OF THE INVENTION

An embodiment of the present invention provides a medical probe including a guidewire, and a connector at a proximal end of the probe. The guidewire is configured to be inserted into an organ of a patient and includes one or more electrical devices fitted at a distal end of the guidewire, and two or more conductors, which connect the electrical devices to a proximal end of the guidewire. The conductors have proximal ends that are exposed at different positions along the proximal end of the guidewire. The connector is fitted with multiple conductive rings that are positioned to mate with the respective exposed proximal ends of the conductors, and to maintain electrical contact with the conductors while the guidewire is rotated.

In some embodiments, when the guidewire is rotated, the exposed proximal ends of the conductors are configured to slide over the respective conductive rings while maintaining the electrical contact.

In some embodiments, the multiple conductors are disposed on an outside of the guidewire.

In an embodiment, the connecter is cylindrically shaped and is further fitted with mating conductors attached to the cylindrical shape, each mating conductor electrically coupled to a respective ring.

In another embodiment, the medical probe further includes one or more visual indicators that are fitted at the proximal end of the probe and are configured to confirm electrical contact between the conductive rings and the exposed proximal ends of the conductors.

In some embodiments, the one or more electrical devices include a sensor.

In some embodiments, the sensor includes a magnetic position sensor.

In an embodiment, the one or more electrical devices include an electrosurgical tool.

In another embodiment, the electrosurgical tool includes a bi-polar electrode.

There is additionally provided, in accordance with another embodiment of the present invention, a system including a medical probe and one or more external modules. The medical probe includes a guidewire, and a connector at a proximal end of the probe. The guidewire is configured to be inserted into an organ of a patient and includes one or more electrical devices fitted at a distal end of the guidewire, and two or more conductors, which connect the electrical devices to a proximal end of the guidewire. The conductors have proximal ends that are exposed at different positions along the proximal end of the guidewire. The connector is fitted with multiple conductive rings that are positioned to mate with the respective exposed proximal ends of the conductors, and to maintain electrical contact with the conductors while the guidewire is rotated. The one or more external modules are connected at the proximal end of the probe to operate the one or more electrical devices.

In some embodiments, the one or more the external modules are configured to operate one or more sensors. In other embodiments, the one or more the external modules are configured to operate one or more electrosurgical tools.

There is further provided, in accordance with another embodiment of the present invention, a method including inserting into an organ of a patient a probe including a guidewire, and a connector at a proximal end of the probe. The guidewire includes one or more electrical devices fitted at a distal end of the guidewire, and two or more conductors, which connect the electrical devices to a proximal end of the guidewire. The conductors have proximal ends that are exposed at different positions along the proximal end of the guidewire. The connector is fitted with multiple conductive rings that are positioned to mate with the respective exposed proximal ends of the conductors, and to maintain electrical contact with the conductors while the guidewire is rotated. The one or more electrical devices are operated using one or more external modules connected at the proximal end of the probe.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a brain procedure performed using a magnetically position-tracked rotatable guidewire, in accordance with an embodiment of the present invention;

FIG. 2 is a schematic, pictorial illustration of the magnetically position-tracked rotatable guidewire of FIG. 1 with an integral connector, in accordance with an embodiment of the present invention; and

FIG. 3 is a flow chart that schematically illustrates a method of using the magnetically position-tracked rotatable guidewire of FIG. 1, in accordance with an embodiment of the present invention.

### DETAILED DESCRIPTION OF EMBODIMENTS

### Overview

Some medical procedures require navigating and manipulating (e.g., rotating) a distal end of a medical probe inside an organ of a patient. For example, guidewires for neurosurgery are used to slide elements used in the surgery, such as an electro-surgical apparatus, into position inside the brain of a patient. These guidewires are normally tracked fluoroscopically, and, typically, rotation of the guidewire is relatively unrestricted. However, fluoroscopy uses a large amount of ionizing radiation, and therefore it is preferable to limit its use. As an alternative, the position and direction of the guidewire can be tracked magnetically, using a magnetic field sensor located at the distal end of the guidewire, with connecting conductors to the proximal end. However, the presence of the conductors may restrict free rotation of the guidewire.

In addition to various type of sensors (e.g., position, pressure, temperature), other electrical devices, such as electrosurgical devices (e.g., a bi-polar tip electrode), may be fitted at the distal edge of the guidewire for therapeutic use, such as electro-cauterizing a wound in brain tissue of the patient, radiofrequency (RF) ablation of cancerous brain tissue, or irreversibly electroporation of cancerous tissue. Therapeutic electrical energy is typically provided as a high-current signal and/or a high-voltage signal, which may harm sensing devices, such as the magnetic sensor, if they share electrical connections. However, multiple separate conductors may further restrict free rotation of the guidewire.

Embodiments of the present invention that are described hereinafter provide improved techniques for electromechanical connectivity to different electrical devices incorporated into the distal end of a guidewire of a probe for insertion into an organ of a patient. To conduct signals to and from the devices (e.g., position sensor and ablation electrode), a set of insulated conductive cabling is disposed on the outside of the guidewire. For example, the embodiments may provide connectivity to one set of insulated conductive cabling for sensors, and further provide separate connectivity to electrosurgical devices, such as a bi-polar tip electrode. One or more sensors may be linked by the same wire pair, using, for example, signal modulation and demodulation techniques. The electrosurgical devices may share conductive cabling, by, for example, switching between electrical therapeutic energy sources in a system using the probe.

In another embodiment, two or more devices share a common line to conduct signals on the line (e.g., use a common ground). Also, some signals may be single-ended. Thus, the actual number of conductors may vary according to the required functionalities of the probe, and in general be two or more of such.

To enable free rotation of the guidewire, terminations of the two different sets of conductive cabling are exposed at different positions along the guidewire at the proximal end of the probe (e.g., of the guidewire). A connector, for example cylindrically shaped one, at the proximal end comprises multiple respective conductive rings (e.g., sets of rings) which are positioned to mate with the exposed conductors when the guidewire slides into the connector (e.g., into the cylindrical shape). The mating of the rings with the exposed wire terminations may be confirmed by any convenient audiovisual means, such as by illuminating LEDs. Each of the conductive ring is further connected by cabling in the connector to an external module to operate the device, such as to a magnetic tracking system to operate a magnetic sensor, and to a high power/voltage therapeutic module, such as an electrical power generator, to drive a bi-polar electrode.

Connecting the guidewire with this type of rotatable connectors means that the guidewire is free to rotate, while still being able to operate with electrical devices mounted on the distal end. The disclosed techniques enable improved safety (e.g., by reducing the use of ionizing radiation) and accessibility of invasive medical tools to target tissue.

### System Description

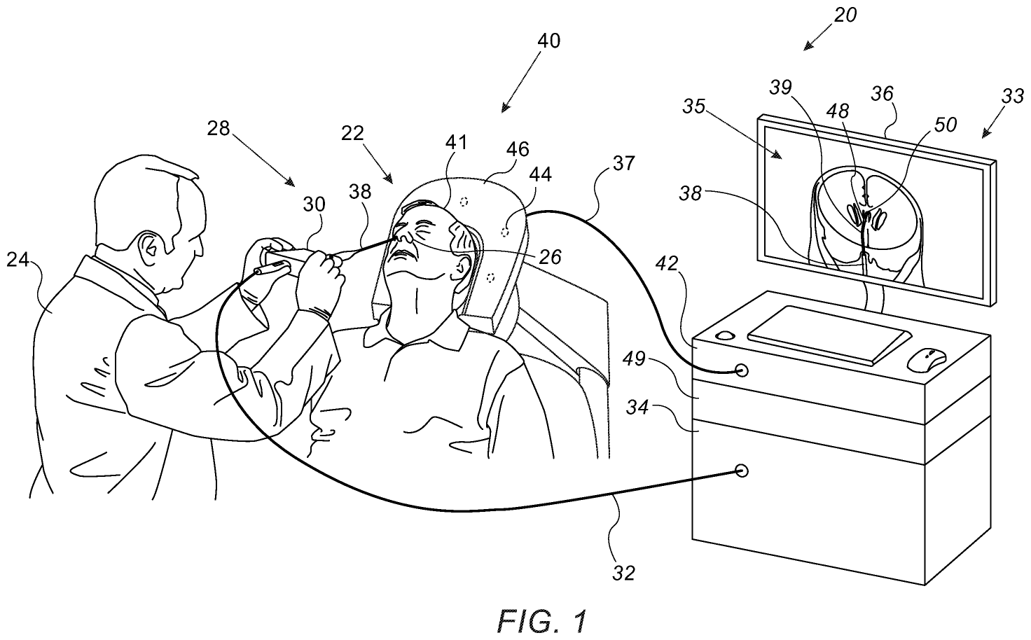

FIG. 1 is a schematic, pictorial illustration of a brain procedure using a magnetically position-tracked rotatable guidewire 39, in accordance with an embodiment of the present invention. In some embodiments, a brain diagnostics and treatment system 20, which comprises surgical apparatus 28, is configured to carry out a brain procedure, such as electro-cauterizing a wound in brain tissue of a patient 22, radiofrequency (RF) ablating cancerous brain tissue, or irreversibly electroporating cancerous tissue.

Surgical apparatus 28 comprises guidewire 39 that is inserted into the brain via an insertion tube 38 and comprising a magnetic position sensor 48 and a bi-polar electrode 50, which a physician 24 inserts into a nose 26 of patient 22 to access brain tissue. Surgical apparatus 28 further comprises a handheld proximal-end assembly 30, coupled to a proximal end of rotatable guidewire 39, which is configured to assist physician 24 to manipulate guidewire 39 in a head 41 of patient 22.

System 20 comprises a magnetic position-tracking system, which is configured to track a position of sensor 48 in the brain. The magnetic position-tracking system comprises a location pad 40, which comprises field generators 44 fixed on a frame 46. In the exemplary configuration shown in FIG. 1, pad 40 comprises five field generators 44, but may alternatively comprise any other suitable number of generators 44. Pad 40 further comprises a pillow (not shown) placed under head 41 of patient 22, such that generators 44 are located at fixed, known positions external to head 41. The position sensor generates position signals in response to sensing external magnetic fields generated by field generators 44, thereby enabling a processor 34 to estimate the position of sensor 48.

This technique of position sensing is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, which prior applications are hereby incorporated by reference in their entirety herein into this application as if set forth in full.

In some embodiments, system 20 comprises a console 33, which comprises a memory 49, and a driver circuit 42 configured to drive field generators 44, via a cable 37, with suitable signals so as to generate magnetic fields in a predefined working volume in space around head 41.

Processor 34 is typically a general-purpose computer, with suitable front end and interface circuits for receiving signals from position sensor 48 and electrically driving bi-polar electrode 50 via a cable 32, and for controlling other components of system 20 described herein.

In some embodiments, processor 34 is configured to receive, via an interface (not shown), one or more anatomical images, such as reference MRI image 35 depicting a two-dimensional (2D) slice of head 41. In the example of FIG. 1, image 35 depicts a sectional coronal view of anterior brain tissue of patient 22. Processor 34 is configured to select one or more slices from the MRI images, to register the position of electrode 50 that is estimated using position sensor 48 with the medical image, and then display the tracked position in image 35 to physician 24 on display 36. Processor 34 is configured to register the position of electrode 50 with image 35 in the coordinate system of the magnetic position-tracking system and/or in a coordinate system of the medical image.

Console 33 further comprises input devices, such as a keyboard and a mouse, for controlling the operation of the console, and user display 36, which is configured to display the data (e.g., images) received from processor 34 and/or to display inputs inserted by a user using the input devices (e.g., by physician 24).

FIG. 1 shows only elements related to the disclosed techniques, for the sake of simplicity and clarity. System 20 typically comprises additional modules and elements that are not directly related to the disclosed techniques, and thus are intentionally omitted from FIG. 1 and from the corresponding description.

Processor 34 may be programmed in software to carry out the functions that are used by the system, and to store data in memory 49 to be processed or otherwise used by the software. The software may be downloaded to the processor in electronic form, over a network, for example, or it may be provided on non-transitory tangible media, such as optical, magnetic or electronic memory media. Alternatively, some or all of the functions of processor 34 may be carried out by dedicated or programmable digital hardware components. In particular, processor 34 runs a dedicated algorithm as disclosed herein, including in FIG. 3, that enables processor 34 to perform the disclosed steps, as further described below.

### Neurosurgery Guidewire with Integral Connector for Sensing and Applying Therapeutic Electrical Energy

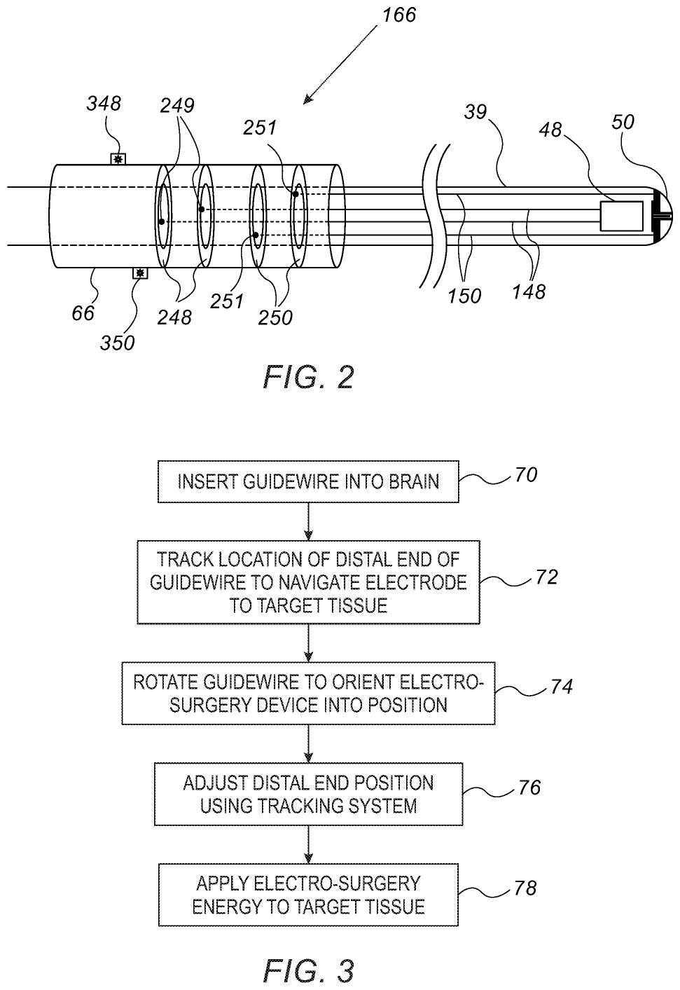

FIG. 2 is a schematic, pictorial illustration of the magnetically position-tracked rotatable guidewire 39 of FIG. 1 with an integral connector 166, in accordance with an embodiment of the present invention. As seen, the rotatable distal end of guidewire 39 comprises a bi-polar electrode 50, and a sensor 48, which in the shown embodiment is a magnetic position sensor. Signals from sensor 48 are received via sensor cabling 148 attached on the outside of guidewire 39. Therapeutic electrical energy, in the form of high electrical current or high voltage, is supplied to electrode 50 via electrode cabling 150 that is also attached on the outside of guidewire 39.

As seen in FIG. 2, termination pairs 249 and 251 of the two sets of conductive cabling, at the proximal end of the guidewire, are exposed at different positions along the guidewire. The guidewire is inserted in a cylinder 66 at a proximal end of the probe, having two sets of respective conductive ring pairs 248 and 250 of integral connector 166 which are positioned to mate with the exposed conductors of integral connector 166. The mating of ring pairs 248 and 250 with the exposed wire terminations 249 and 251 may be confirmed by any convenient means, such as by illuminating LEDs 348 and 350, respectively. Conductive ring pairs 248 and 250 are each connected by cabling (not shown) in cylinder 66 to a magnetic tracking system, and to an electrical power generator, respectively.

The configuration in FIG. 2 is depicted by way of example for the sake of conceptual clarity. In other embodiments, integral connector 166 may include additional elements, such as, for example, elastic elements that press termination pairs 249 and 251 against conductive ring pairs 248 and 250, respectively, to ensure robustness of electrical contact.

FIG. 3 is a flow chart that schematically illustrates a method of using the magnetically position-tracked rotatable guidewire 39 of FIG. 1, in accordance with an embodiment of the present invention. The process begins with physician 24 inserting rotatable guidewire 39 through trocar 38 into the brain of patient 22, at a guidewire insertion step 70. Next, physician 24 operates system 20 to magnetically track a location in the brain of the distal end of guidewire 39, e.g., of electrode 50, at a guidewire position tracking step 72, to advance electrode 50 in a direction of target tissue. Using the disclosed integral connector, the system continues to receive signals from sensor 48 even when the guidewire is rotated in the process of advancing the guidewire.

At guidewire rotation step 74, physician 24 rotates guidewire 39 to orient an electrosurgery device, such as electrode 50, into a suitable position to treat target tissue. Rotation step 74 and advancement step 72 of the guidewire may be performed multiple times, until the physician is satisfied with the position of electrode 50 relative to target tissue.

Physician 24 may further improve the quality of engagement of electrode 50 with target tissue by adjusting the distal end position using the tracked location (e.g., using the tracked position registered with medical image 35), at a position adjustment step 76.

Finally, when ready, physician 24 applies therapeutic electrosurgery energy to tissue using electrode 50, at an electrosurgery treatment step 78. Using the disclosed integral connector, the system can continue to apply the therapeutic electrosurgery energy to electrode 50 even when the guidewire is rotated in the process.

The example flow chart shown in FIG. 3 is chosen purely for the sake of conceptual clarity. In alternative embodiments physician 24 may perform additional steps, such as employing additional monitoring steps (e.g., fluoroscopy) to verify the successful outcome of the procedure, and/or may apply other sensors fitted to distal end 31, for example, to acquire additional clinical data, such as intracranial pressure. In general, multiple sensors may be linked by the same wire pair, using for example, signal modulation and demodulation techniques.

Although the embodiments described herein mainly address brain procedures, the methods and systems described herein can also be used in other applications that require guiding a medical device in other organs, such as located in the abdomen or the chest.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical probe, comprising:
a guidewire which is configured to be inserted into an organ of a patient and comprises:
one or more electrical devices fitted at a distal end of the guidewire;
two or more conductors, which connect the electrical devices to a proximal end of the guidewire, the conductors having proximal ends that are exposed at different positions along the proximal end of the guidewire; and
a connector at a proximal end of the probe, which is fitted with multiple conductive rings that are positioned to mate with the respective exposed proximal ends of the conductors, and to maintain electrical contact with the conductors while the guidewire is rotated.

2. The medical probe according to claim 1, wherein, when the guidewire is rotated, the exposed proximal ends of the conductors are configured to slide over the respective conductive rings while maintaining the electrical contact.

3. The medical probe according to claim 1, wherein the multiple conductors are disposed on an outside of the guidewire.

4. The medical probe according to claim 1, wherein the connecter is cylindrically shaped and is further fitted with mating conductors attached to the cylindrical shape, each mating conductor electrically coupled to a respective ring.

5. The medical probe according to claim 1, further comprising one or more visual indicators that are fitted at the proximal end of the probe and are configured to confirm electrical contact between the conductive rings and the exposed proximal ends of the conductors.

6. The medical probe according to claim 1, wherein the one or more electrical devices comprise a sensor.

7. The medical probe according to claim 6, wherein the sensor comprises a magnetic position sensor.

8. The medical probe according to claim 1, wherein the one or more electrical devices comprise an electrosurgical tool.

9. The medical probe according to claim 8, wherein the electrosurgical tool comprises a bi-polar electrode.

10. A system, comprising:
a medical probe, comprising:
a guidewire which is configured to be inserted into an organ of a patient and comprises:
one or more electrical devices fitted at a distal end of the guidewire;
two or more conductors, which connect the electrical devices to a proximal end of the guidewire, the conductors having proximal ends that are exposed at different positions along the proximal end of the guidewire; and
a connector at a proximal end of the probe, which is fitted with multiple conductive rings that are positioned to mate with the respective exposed proximal ends of the conductors, and to maintain electrical contact with the conductors while the guidewire is rotated; and
one or more external modules connected at the proximal end of the probe to operate the one or more electrical devices.

11. The system according to claim 10, wherein one or more the external modules are configured to operate one or more sensors.

12. The system according to claim 11, wherein the one or more sensors comprise a magnetic position sensor.

13. The system according to claim 10, wherein one or more the external modules are configured to operate one or more electrosurgical tools.

14. The system according to claim 13, wherein the one or more electrosurgical tools comprise a bi-polar electrode.

15. A method, comprising:
inserting into an organ of a patient a probe comprising:
a guidewire which is configured to be inserted into an organ of a patient and comprises:
one or more electrical devices fitted at a distal end of the guidewire;
two or more conductors, which connect the electrical devices to a proximal end of the guidewire, the conductors having proximal ends that are exposed at different positions along the proximal end of the guidewire; and
a connector at a proximal end of the probe, which is fitted with multiple conductive rings that are positioned to mate with the respective exposed proximal ends of the conductors, and to maintain electrical contact with the conductors while the guidewire is rotated; and
operating the one or more electrical devices using one or more external modules connected at the proximal end of the probe.

16. The method according to claim 15, wherein operating the electrical devices comprises operating one or more sensors.

17. The method according to claim 16, wherein the one or more sensors comprise a magnetic position sensor.

18. The method according to claim 15, wherein operating the electrical devices comprises operating one or more electrosurgical tools.

19. The method according to claim 18, wherein the one or more electrosurgical tools comprise a bi-polar electrode.
